(19) United States
(12) Patent Application Publication
(10) Pub. No.: US 2009/0202584 A1
(43) Pub. Date: Aug. 13, 2009

(54) TREATMENT OF EPSTEIN-BARR VIRUS-ASSOCIATED DISEASES

(75) Inventors: Scott Anthony Thomson, Queensland (AU); Jai Kumar Duraiswamy, Queensland (AU); Denis James Moss, Oueensland (AU)

Correspondence Address: KNOBBE MARTENS OLSON & BEAR LLP, 2040 MAIN STREET, FOURTEENTH FLOOR, IRVINE, CA, 92614, US

(73) Assignee: Savine Therapeutics Pty, Eastwood, NSW (AU)

(21) Appl. No.: 12/096,296

(22) PCT Filed: Dec. 6, 2006
(86) PCT No.: PCT/AU06/01854

§ 371 Date: Mar. 17, 2009

(30) Foreign Application Priority Data
Dec. 6, 2005 (AU) 2005906851

(51) Int. Cl. A61K 39/385; C07K 14/00; C12N 15/11; C07H 1/00

(52) U.S. Cl. 424/196.11; 530/350; 536/23.72; 536/25.3

(57) ABSTRACT

The present invention relates to a vaccine for the treatment or prevention of an EBV-associated disease in a subject, wherein said vaccine comprises a synthetic polypeptide comprising a plurality of different segments of at least one parent EBV polypeptide, and wherein the segments are linked together in a different relationship relative to their linkage in the at least one parent EBV polypeptide.

### TECHNICAL FIELD

The present invention relates to methods, vaccines, immunological compositions and synthetic polypeptides for treating and/or preventing Epstein-Barr Virus (EBV)-associated diseases, and to associated methods for modulating an immune response.

### BACKGROUND ART

The lack of a safe and efficient vaccine strategy that can provide substantially complete immunological coverage against EBV-associated diseases is an important problem, and one that has prevented progress in treatments for several EBV-associated diseases such as post-transplant lymphoproliferative disease (PTLD), nasopharyngeal carcinoma (NPC) and Hodgkin's lymphoma (HL).

For each of these diseases, cytotoxic T lymphocytes (CTL) are an important effector mechanism in control of EBV infection, and the possibility of immunological intervention in ongoing EBV-associated malignancy has been considerably enhanced in recent years by the observation that adoptive transfer of EBV-specific CTL activated in vitro by autologous lymphoblastoid cell lines can be used to treat PTLD which occasionally arise in graft recipients. In this instance, the CTL bulk cultures that are adoptively transferred are dominated by effector cells with specificity towards the immunodominant EBV nuclear proteins, EBNAs 3, 4 and 6.

However, the option of extending this strategy for application to NPC and HL has been hampered by the more limited range of potential virus-encoded targets expressed in these malignancies, namely EBNA1, LMP1 and LMP2. Of these, LMP1 and LMP2 are the only potential targets, because EBNA1 is poorly processed and poorly presented by virus-infected cells through the MHC class I pathway.

Further difficulties in formulating new treatments for NPC and HL arise due to the limited possibility of using LMP1 to expand effector cells for adoptive transfer because of the low precursor frequency to these epitopes in healthy individuals. Moreover, the use of full-length LMP proteins in a clinical setting is hampered since these proteins can independently initiate an oncogenic process in normal cells.

The present invention is predicated on the surprising and unexpected finding that EBV-associated diseases can be treated and/or prevented using a scrambled antigen vaccine, or “SAVINE”.

### SUMMARY OF THE INVENTION

According to a first aspect of the present invention, there is provided a vaccine for the treatment or prevention of an EBV-associated disease in a subject, wherein said vaccine comprises a synthetic polypeptide comprising a plurality of different segments of at least one parent EBV polypeptide, and wherein the segments are linked together in a different relationship relative to their linkage in the at least one parent EBV polypeptide.

The at least one parent EBV polypeptide may be selected from the group including EBNA1, LMP1 and LMP2.

The EBV-associated disease may be cancer.

The cancer may be selected from the group including nasopharyngeal carcinoma (NPC), Hodgkin's lymphoma (HL) and post-transplant lymphoproliferative disease (PTLD).

The synthetic polypeptide may consist essentially of different segments of a single parent EBV polypeptide.

Alternatively, the synthetic polypeptide may consist essentially of different segments of a plurality of different parent EBV polypeptides.

The segments in said synthetic polypeptide may be linked sequentially in a different order or arrangement relative to that of corresponding segments in said at least one parent EBV polypeptide.

At least one of said segments may comprise partial sequence identity or homology to one or more other said segments. The sequence identity or homology may be contained at one or both ends of said at least one segment.

According to a second aspect of the present invention, there is provided a synthetic polypeptide, wherein said polypeptide comprises a plurality of different segments of at least one parent EBV polypeptide, and wherein the segments are linked together in a different relationship relative to their linkage in the at least one parent EBV polypeptide.

According to a third aspect of the present invention, there is provided a synthetic polynucleotide encoding the synthetic polypeptide of the second aspect.

The synthetic polypeptide may comprise the sequence as set forth at SEQ ID NO:1.

According to a fourth aspect of the present invention, there is provided a synthetic construct comprising the polynucleotide of the third aspect operably linked to a regulatory polynucleotide.

According to a fifth aspect of the present invention, there is provided a method for producing the synthetic polynucleotide of the third aspect, comprising linking together in the same reading frame a plurality of nucleic acid sequences encoding different segments of at least one parent EBV polypeptide to form a synthetic polynucleotide whose sequence encodes said segments linked together in a different relationship relative to their linkage in the at least one parent EBV polypeptide.

The method may further comprise fragmenting the sequence of a respective parent EBV polypeptide into fragments and linking said fragments together in a different relationship relative to their linkage in said parent EBV polypeptide sequence.

The fragments may be randomly linked together.

The method may further comprise reverse translating the sequence of a respective parent EBV polypeptide or a segment thereof to provide a nucleic acid sequence encoding said parent EBV polypeptide or said segment.

An amino acid of said parent EBV polypeptide sequence may be reverse translated to provide a codon which has higher translational efficiency than other synonymous codons in a cell of interest.

Additionally or alternatively, an amino acid of said parent EBV polypeptide sequence may be reverse translated to provide a codon which, in the context of adjacent or local sequence elements, has a lower propensity of forming an undesirable sequence that is refractory to the execution of a task.

The undesirable sequence may be a palindromic sequence or a duplicated sequence.

The task may be cloning, sequencing, enhancing the stability of the polynucleotide or enhancing in vivo translation.

According to a sixth aspect of the present invention, there is provided a composition comprising an immunopotentiating agent selected from the group consisting of the vaccine of the first aspect, the synthetic polypeptide of the second aspect, the synthetic polynucleotide of the third aspect and the synthetic construct of the fourth aspect, together with a pharmaceutically acceptable carrier.

The composition may optionally comprise an adjuvant.

According to a seventh aspect of the present invention, there is provided a method for modulating an immune response, which response is directed against an EBV-associated disease, comprising administering to a patient in need of such treatment an effective amount of an immunopotentiating agent selected from the group consisting of the vaccine of the first aspect, the synthetic polypeptide of the second aspect, the synthetic polynucleotide of the third aspect, the synthetic construct of the fourth aspect, or the composition of the sixth aspect.

According to an eighth aspect of the present invention, there is provided a method for treatment and/or prophylaxis of an EBV-associated disease, comprising administering to a patient in need of such treatment an effective amount of an immunopotentiating agent selected from the group consisting of the vaccine of the first aspect, the synthetic polypeptide of the second aspect, the synthetic polynucleotide of the third aspect, the synthetic construct of the fourth aspect, or the composition of the sixth aspect.

According to a ninth aspect of the present invention, there is provided use of the vaccine of the first aspect, the synthetic polypeptide of the second aspect, the synthetic polynucleotide of the third aspect, the synthetic construct of the fourth aspect and the composition of the sixth aspect for the modulation of an immune response.

According to a tenth aspect of the present invention, there is provided use of the vaccine of the first aspect, the synthetic polypeptide of the second aspect, the synthetic polynucleotide of the third aspect, the synthetic construct of the fourth aspect and the composition of the sixth aspect for the manufacture of a medicament for the treatment of an EBV-associated disease.

According to an eleventh aspect of the present invention, there is provided a vaccine comprising the synthetic polypeptide of the second aspect, the synthetic polynucleotide of the third aspect, the synthetic construct of the fourth aspect or the composition of the sixth aspect for use in the treatment of an EBV-associated disease.

### BRIEF DESCRIPTION OF THE DRAWINGS

The present invention will now be described, by way of example only, with reference to the following drawings.

FIG. 1. Schematic representation of NPC SAVINE that encodes overlapping peptide sets spanning LMP1, LMP2 and EBNA1 proteins randomly joined together. The DNA sequence encoding these 3 proteins was constructed using sequence-specific overlapping oligonucleotides varying in length from 20 to 100 bp. Sequences were joined together by stepwise asymmetric PCR to create subcassettes. These subcassettes were joined together using restriction digestion and PCR to develop the final NPC SAVINE construct of 6.8 kb. This construct was then cloned into replication deficient adenovirus vector (Ad5F35). The recombinant adenovirus (AdSAVINE) expressing SAVINE construct was obtained by transfecting into HEK293 cells. This SAVINE construct was also inserted into vaccinia and fowl pox virus delivery vectors (see Thomson S. A., Jaramillo A. B., Shoobridge M., Dunstan K. J., Everett B., Ranasinghe C., Kent S. J., Gao K., Medveckzy C. J., French R. A., Ramshaw I. A. Development Of A Synthetic Consensus Sequence Scrambled Antigen HIV-1 Vaccine Designed for Global Use (2005) Vaccine, 23(38) 4647-57).

FIG. 2. Processing and presentation of defined epitopes within SAVINE construct. LMP1, LMP2 and EBNA1-peptide specific CTL kill targets infected with SAVINE. The defined epitope-specific CTL polyclonal lines or CTL clones within EBNA1 (HPV, HLA-B35 restricted), LMP1 (YLL and YLQ, HLA A2-restricted; IAL, HLA B35-restricted) and LMP2 (CLG, LTA and LLS, HLA A2-restricted; PYL, HLA-A23-restricted; IED, HLA-B40-restricted) antigens were generated from four EBV seropositive healthy donors. The specificity of these CTL was tested against the defined epitope-loaded PHA blasts in a cytolytic assay. Subsequently, to find out whether the defined epitopes within EBNA1, LMP1 and LMP2 antigens were endogenously processed, HLA-matched fibroblasts were first infected with vaccinia, fowl pox or adenovirus vectors expressing SAVINE construct (MOI, 10:1). The target fibroblasts infected with vaccinia TK-, empty adenovirus or uninfected fibroblasts were used as controls. These targets were then tested for the cytolytic activity against EBNA1, LMP1 and LMP2 epitope-specific CTL polyclonal lines or CTL clones generated from EBV seropositive healthy donors in a Chromium release assay. An Effector:Target ratio of 10:1 is used in these assays. HLA-matched fibroblasts infected with either vaccinia, fowl pox or adenovirus vectors expressing SAVINE construct showed cytolytic activity, whereas fibroblasts infected with control vectors were not lysed. These results demonstrate that the defined epitopes in the SAVINE construct are processed and presented to the targets cells very efficiently.

FIG. 3. Activation of SAVINE and LCL stimulated CTL from EBV seropositive healthy donors. (A) and (B) PBMCs from healthy human EBV carriers (ScBu and DoSc) were stimulated with autologous PBMCs infected (responder to stimulator ratio of 2:1) with either AdSAVINE, AdPoly or autologous LCL (30:1). All cultures were restimulated at weekly intervals using γ-irradiated autologous LCLs infected as described. Three days after 3 restimulations the cultured cells were used as effectors in a Chromium release assay against peptide-sensitized autologous PHA blasts. (C) The cultured cells were also tested by ELISPOT and the results are expressed as spot forming cells (SFC) per 10⁶ CTL.

FIG. 4. Mapping of EBNA1, LMP1 and LMP2-specific responses in EBV seropositive healthy donors. The amino acid sequences of full length LMP1 antigen were derived from both Asian EBV strain, CAO (32 peptides of 17 mer in length overlapping by 8 residues) and Caucasian prototype 1 EBV strain, B95.8 (42 peptides of 17 mer in length overlapping by 8 residues). The amino acid sequences of full length LMP2 (49 peptides of 20 mer in length overlapping by 10 residues) and EBNA1 (69 peptides of 15 mer in length overlapping by 10 residues) antigens were derived from Caucasian prototype 1 EBV strain, B95.8. Adenovirus-SAVINE and LCL-activated CTL generated from four EBV seropositive healthy donors were tested for the secretion of IFN-γ after stimulation with overlapping peptides. Specific T cell reactivity to defined CD8⁺ as well as CD4⁺ T cell epitopes were observed. In addition to reactivity against already defined peptides, four of these new peptide pool sequences (2 each from LMP1 and LMP2) showed reactivity by both SAVINE and LCL-activated CTL and four of these new peptide pool sequences (1 each from CAO LMP1, B95.8 LMP1 LMP2 and EBNA1) showed reactivity by SAVINE activated CTL.

FIG. 5. Ex vivo ELISPOT analysis of specific CTL after priming with Ad SAVINE and boosting with Vaccinia SAVINE or Fowl pox SAVINE. Two groups of HLA-A2/Kb transgenic mice (n=5) were immunised s.c. with Ad SAVINE (10⁹ PFU) and two weeks later, these mice were again injected with either Vaccinia-SAVINE (10⁷ PFU) or Fowl pox SAVINE (2×10⁷ PFU). Two weeks later, the spleen cells were harvested and CTL response was assessed by ELISPOT assays and the results are expressed as mean+SE of spot-forming cells (SFC) per 10⁶ splenocytes.

FIG. 6. Therapeutic adoptive transfer of in-vitro expanded SAVINE-CTL from spleen cells of HLA transgenic mice primed with adeno-SAVINE and boosted with Vaccinia or fowlpox SAVINE cause regression of human NPC. Immunodeficient nude mice were inoculated with human NPC allografts and when the tumour size was approximately 0.2 cm³ in size (14 days after tumour inoculation), each group of tumour-bearing nude mice (n=6 mice/group) was adoptively transferred with either 5×10⁶ Ad (primed)-VV (boosted) SAVINE-specific T cells or 5×10⁶ Ad-FPV SAVINE-specific T cells. Another group of nude mice was injected with 5×10⁶ Ad-FPV SAVINE-CTL and treated with human IL-15 (5 μg) injection i.p. 1, 2 and 3 days after each adoptive transfer. Control groups included were mice injected with 5×10⁶ LMP polyepitope-specific CTL, cytomegalovirus polyepitope (CMV)-specific CTL, CD8 depleted Ad-FPV SAVINE-CTL or untreated. The therapeutic efficacy of SAVINE-specific T cells was assessed by regular monitoring of tumour regression and mice showing a tumour size of >1.0 cm³ in size were sacrificed. Untreated mice, mice that received CMV T cells or CD8 depleted Ad-FPV SAVINE-CTL did not result in inhibition of tumour growth and the tumours in these mice reached 1.0 cm³ by about 12-24 days after the first T cell transfer. Mice receiving CD8 depleted LMP-CTL were sacrificed by about 12-78 days after first CTL transfer. After 90 days, 1/6 mice receiving either Ad-FPV SAVINE-CTL alone or mice receiving Ad-FPV SAVINE-CTL as well as IL15 sustained regression and the regression in 2/6 mice sustained in mice that received Ad-VV SAVINE-CTL.

### DEFINITIONS

As used herein, the term “comprising” means “including principally, but not necessarily solely”. Furthermore, variations of the word “comprising”, such as “comprise” and “comprises”, have correspondingly varied meanings.

As used herein the terms “treating” and “treatment” refer to any and all uses which remedy a condition or symptoms, prevent the establishment of a condition or disease, or otherwise prevent, hinder, retard, or reverse the progression of a condition or disease or other undesirable symptoms in any way whatsoever.

As used herein the term “effective amount” includes within its meaning a non-toxic but sufficient amount of an agent or compound to provide the desired effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein, the terms “polypeptide”, “peptide” and “protein” are used interchangeably to refer to a polymer of amino acid residues and to fragments, variants, analogues, orthologues or homologues thereof. Thus, these terms apply both to amino acid polymers in which one or more amino acid residues is a synthetic non-naturally occurring amino acid, such as a chemical analogue of a corresponding naturally occurring amino acid, as well as to naturally-occurring amino acid polymers.

As used herein, the term “polynucleotide” or “nucleic acid” designates oligonucleotides comprising mRNA, RNA, cRNA, cDNA or DNA or combinations thereof.

As used herein, the term “operably linked” refers to transcriptional and translational regulatory polynucleotides that are positioned relative to a polypeptide-encoding polynucleotide in such a manner such that the polynucleotide is transcribed and the polypeptide is translated.

As used herein, the term “synthetic polypeptide” refers to a polypeptide formed in vitro by the manipulation of a polypeptide or corresponding polynucleotide into a form not normally found in nature. For example, a synthetic polypeptide may be the translational product of a synthetic polynucleotide.

As used herein, the term “synthetic polynucleotide” refers to a polynucleotide formed in vitro by the manipulation of a polynucleotide into a form not normally found in nature. For example, the synthetic polynucleotide can be in the form of an expression vector. Generally, such expression vectors include transcriptional and translational regulatory polynucleotides operably linked to the polynucleotide.

As used herein, the term “EBV-associated disease” means any disease, disease state or disorder caused by or associated with Epstein-Barr Virus (EBV), including but not limited to cancer, such as nasopharyngeal carcinoma, Hodgkin's lymphoma or post-transplant lymphoproliferative disease.

As used herein, the term “parent EBV polypeptide” means a polypeptide that has been isolated or derived from Epstein-Barr Virus (EBV), or which is homologous thereto, and used to produce a synthetic polypeptide. The parent EBV polypeptide may be an EBV polypeptide encoded by a naturally occurring gene. Alternatively, parent EBV polypeptide may be an EBV polypeptide that is not naturally occurring but has been engineered using recombinant techniques. In this instance, a polynucleotide encoding the parent polypeptide may comprise different but synonymous codons relative to a natural gene encoding the same polypeptide. Alternatively, the parent EBV polypeptide may not correspond to a natural polypeptide sequence. For example, the parent EBV polypeptide may comprise one or more consensus sequences common to a plurality of polypeptides.

As used herein, the term “modulating” means increasing or decreasing, either directly or indirectly, an immune response against an antigen.

As used herein, the term “conservative amino acid substitution” refers to a substitution or replacement of one amino acid for another amino acid with similar properties within a polyepitope chain (primary sequence of a protein). For example, the substitution of the charged amino acid glutamic acid (Glu) for the similarly charged amino acid aspartic acid (Asp) would be a conservative amino acid substitution.

Within the scope of the terms “protein”, “polypeptide”, “polynucleotide” and “nucleic acid” as used herein are fragments and variants thereof, including but not limited to reverse compliment and antisense forms of polynucleotides and nucleic acids.

The term “fragment” refers to a polynucleotide or polypeptide sequence that encodes a constituent or is a constituent of a full-length protein or gene. In terms of the polypeptide the fragment possesses qualitative biological activity in common with the full-length protein.

The term “variant” as used herein refers to substantially similar sequences. Generally, nucleic acid sequence variants encode polypeptides which possess qualitative biological activity in common. Generally, polypeptide sequence variants also possess qualitative biological activity in common. Further, these polypeptide sequence variants may share at least 50%, 55%, 60%, 65%, 70%, 75%, 80%, 85%, 90%, 95%, 96%, 97%, 98% or 99% sequence identity.

Further, a variant polypeptide may include analogues, wherein the term “analogue” means a polypeptide which is a derivative of the disclosed polypeptides, which derivative comprises addition, deletion or substitution of one or more amino acids, such that the polypeptide retains substantially the same function as the native polypeptide from which it is derived.

### BEST MODE OF PERFORMING THE INVENTION

A recent technology platform referred to as SAVINE (“scrambled antigen vaccine”) as disclosed in WO 01/90197 (the disclosure of which is incorporated herein by reference) has been applied by the inventors in relation to novel treatments for Epstein-Barr Virus (EBV)-associated diseases such as nasopharyngeal carcinoma (NPC), Hodgkin's lymphoma (HL) and post-transplant lymphoproliferative disease (PTLD).

Particular difficulties associated with traditional EBV treatment regimes include the fact that only 3 EBV antigens are expressed in EBV-derived NPC cells, being EBNA1, LMP1 and LMP2. The ability to selectively target EBV tumour cells is therefore very limited. In addition, of the 3 expressed antigens, EBNA1 is poorly presented on the surface of EBV infected cells and/or the progeny of such cells, and full-length LMP proteins cannot be used to induce appropriate CTL immune responses as such proteins can be independently oncogenic. Further, the use of LMP1 to expand effector cells for treatment regimes employing adoptive T cell transfer is limited because of low frequency of precursor cells specific for LMP epitopes. Indeed, EBV-specific CTL populations that have been activated in vitro for adoptive transfer are often dominated by CTLs specific for EBV nuclear proteins rather than the cell surface antigens EBNA1, LMP1 and LMP2.

Innovation beyond traditional treatment regimes such as chemotherapy and radiotherapy has therefore been difficult to progress in relation to EBV-associated diseases. Indeed, present treatments for EBV-associated diseases such NPC and HL based on radiotherapy and chemotherapy are only partially successful and involve significant side effects. Significantly, the lack of a vaccine-based approach in relation to EBV has meant a lack of any preventative/prophylactic measures.

Accordingly, although EBV infects over 95% of the world's population, current treatment protocols such as radiotherapy and chemotherapy for the EBV-associated disease nasopharyngeal carcinoma (NPC) provide only 5 year survival to about 80% of patients, with late morbidity also a major concern.

In order to overcome such difficulties, the inventors have developed a vaccination regime not only for the treatment but also for the prevention/prophylaxis of EBV-associated diseases. The inventors have scrambled DNA sequence drawn from the EBV cell-surface expressed EBV antigens EBNA1, LMP1 and LMP2 in overlapping 30 amino acid sequences (overlapping by 15 amino acids). This SAVINE sequence has been inserted into a replication-deficient adenovirus vector based on adenovirus 5 with a fibre protein from adenovirus 35 (Ad5F35).

This scrambled antigen vaccine approach has been employed as a novel means for potential treatment of EBV-associated diseases. Accordingly, the invention disclosed herein demonstrates (1) that a scrambled DNA sequence drawn from the EBV antigens EBNA1, LMP1 and LMP2 inserted into the viral vector Ad5F35 is able to be efficiently processed and presented to antigen-specific T cells, (2) that a SAVINE-specific CTL response can be elicited from EBV immune subjects, (3) that the CTL (priming) response can be boosted by subsequent immunization with a vaccinia or fowlpox SAVINE construct, and that (4) prime-boosted SAVINE CTL which are then expanded in vitro using defined epitope CTL peptides can elicit activation of splenocytes in vivo which resist NPC tumour cell growth.

This SAVINE construct therefore has the significant advantage of removing the oncogenic capacity of LMP1 whilst at the same time allowing presentation of all of the possible MHC class I and class II epitopes within EBNA1, LMP1 and LMP2. Furthermore, in its present form, all of the glycine/alanine repeat sequences within EBNA1 have been eliminated, thus minimizing immune inhibitory signals that compromise T cell processing of the entire protein.

Accordingly, the present invention provides vaccines for the treatment or prevention of an EBV-associated disease in a subject, wherein said vaccines comprise a synthetic polypeptide comprising a plurality of different segments of at least one parent EBV polypeptide, and wherein the segments are linked together in a different relationship relative to their linkage in the at least one parent EBV polypeptide.

The at least one parent EBV polypeptide may be selected from the group including EBNA1, LMP1 and LMP2.

The EBV-associated disease may be cancer.

The cancer may be selected from the group including nasopharyngeal carcinoma, Hodgkin's lymphoma and post-transplant lymphoproliferative disease.

Persons of skill in the art will readily appreciate that the synthetic polypeptide may consist essentially of different segments of a single parent EBV polypeptide, or alternatively, the synthetic polypeptide may consist essentially of different segments of a plurality of different parent EBV polypeptides.

It will also be apparent to skilled artisans that the segments in said synthetic polypeptide may be linked sequentially in a different order or arrangement relative to that of corresponding segments in said at least one parent EBV polypeptide.

At least one of said segments may comprise partial sequence identity or homology to one or more other said segments. The sequence identity or homology may be contained at one or both ends of said at least one segment.

### Synthetic Polypeptides

The inventors have been able to disrupt the structure of parent EBV polypeptides sufficiently to impede, abrogate or otherwise alter at least one function of the parent EBV polypeptides, while simultaneously minimising the destruction of potentially useful epitopes that are present in the parent EBV polypeptides, by fusing, coupling or otherwise linking together different segments of the parent EBV polypeptides in a different relationship relative to their linkage in the parent EBV polypeptides. As a result of this change in relationship, the sequence of the linked segments in the resulting synthetic polypeptide is different to a sequence contained within the parent EBV polypeptides.

Accordingly, present invention provides a synthetic polypeptides, wherein said polypeptides comprise a plurality of different segments of at least one parent EBV polypeptide, and wherein the segments are linked together in a different relationship relative to their linkage in the at least one parent EBV polypeptide.

In accordance with the present invention, fusion proteins may also be engineered to improve characteristics of a polypeptide or a variant or fragment thereof. For example, peptide moieties may be added to the polypeptide to increase stability of the polypeptide. The addition of peptide moieties of polypeptides are routine techniques well known to those of skill in the art.

The synthetic polypeptides of the invention are useful as immunopotentiating agents, and are referred to elsewhere in the specification as scrambled antigen vaccines, super attenuated vaccines or “SAVINES”.

Persons of skill in the art will appreciate it is preferable but not essential that the segments in said synthetic polypeptide are linked sequentially in a different order or arrangement relative to that of corresponding segments in said at least one parent EBV polypeptide. For example, in the case of a parent EBV polypeptide that comprises 4 contiguous or overlapping segments A-B-C-D, these segments may be linked in 23 other possible orders to form a synthetic polypeptide. These orders may be selected from the group consisting of: A-B-D-C, A-C-B-D, A-C-D-B, A-D-B-C, A-D-C-B, B-A-C-D, B-A-D-C, B-C-A-D, B-C-D-A, B-D-A-C, B-D-C-A, C-A-B-D, C-A-D-B, C-B-A-D, C-B-D-A, C-D-A-B, C-D-B-A, D-A-B-C, D-A-C-B, D-B-A-C, D-B-C-A, D-C-A-B, and D-C-B-A. Although the rearrangement of the segments is preferably random, it is especially preferable to exclude or otherwise minimise rearrangements that result in complete or partial reassembly of the parent sequence (e.g., ADBC, BACD, DABC). It will be appreciated, however, that the probability of such complete or partial reassembly diminishes as the number of segments for rearrangement increases.

The order of the segments is suitably shuffled, reordered or otherwise rearranged relative to the order in which they exist in the parent EBV polypeptide so that the structure of the polypeptide is disrupted sufficiently to impede, abrogate or otherwise alter at least one function associated with the parent EBV polypeptide. Preferably, the segments of the parent EBV polypeptide are randomly rearranged in the synthetic polypeptide.

The parent EBV polypeptide is suitably a polypeptide that is associated with a disease or condition. For example, the parent polypeptide may be a polypeptide expressed either by EBV, or by a cancer cell caused by, resulting from or associated with an EBV infection. In particular, the parent EBV polypeptide may be selected form the group comprising EBNA1, LMP1 and LMP2.

Treatment of any cancer or tumour caused by, resulting from or associated with EBV is contemplated by the present invention. For example, the cancer or tumour includes, but is not restricted to, post transplant lymphoproliferative disease (PTLD), Hodgkin's Lymphoma and nasopharyngeal carcinoma (NPC).

In a preferred embodiment, the segments are selected on the basis of size. A segment according to the invention may be of any suitable size that can be utilised to elicit an immune response against an antigen encoded by the parent EBV polypeptide. A number of factors can influence the choice of segment size. For example, the size of a segment should be preferably chosen such that it includes, or corresponds to the size of, T cell epitopes and their processing requirement. Practitioners in the art will recognise that class I-restricted T cell epitopes can be between 8 and 10 amino acids in length and if placed next to unnatural flanking residues, such epitopes can generally require 2 to 3 natural flanking amino acids to ensure that they are efficiently processed and presented. Class II-restricted T cell epitopes can range between 12 and 25 amino acids in length and may not require natural flanking residues for efficient proteolytic processing although it is believed that natural flanking residues may play a role. Another important feature of class II-restricted epitopes is that they generally contain a core of 9-10 amino acids in the middle which bind specifically to class II MHC molecules with flanking sequences either side of this core stabilising binding by associating with conserved structures on either side of class II MHC antigens in a sequence independent manner (Brown J. H., Jardetsky T. S., Gorga J. C., Stern L. J., Urban R. G., Strominger J. L., Wiley D. C.: Three-dimensional structure of the human class II histocompatibility antigen HLA-DR1. Nature 1993, 364:33-39). Thus the functional region of class II-restricted epitopes is typically less than 15 amino acids long. The size of linear B cell epitopes and the factors effecting their processing, like class II-restricted epitopes, are quite variable although such epitopes are frequently smaller in size than 15 amino acids. From the foregoing, it is preferable, but not essential, that the size of the segment is at least 4 amino acids, preferably at least 7 amino acids, snore preferably at least 12 amino acids, more preferably at least 20 amino acids and more preferably at least 30 amino acids. Suitably, the size of the segment is less than 2000 amino acids, more preferably less than 1000 amino acids, more preferably less than 500 amino acids, more preferably less than 200 amino acids, more preferably less than 100 amino acids, more preferably less than 80 amino acids and even more preferably less than 60 amino acids and still even more preferably less than 40 amino acids. In this regard, it is preferable that the size of the segments is as small as possible so that the synthetic polypeptide adopts a functionally different structure relative to the structure of the parent EBV polypeptide. It is also preferable that the size of the segments is large enough to minimise loss of T cell epitopes. In an especially preferred embodiment, the size of the segment is about 30 amino acids.

An optional spacer may be utilised to space adjacent segments relative to each other. Accordingly, an optional spacer may be interposed between some or all of the segments. The spacer suitably alters proteolytic processing and/or presentation of adjacent segment(s). In a preferred embodiment of this type, the spacer promotes or otherwise enhances proteolytic processing and/or presentation of adjacent segment(s). Preferably, the spacer comprises at least one amino acid. The at least one amino acid is suitably a neutral amino acid. The neutral amino acid is preferably alanine. Alternatively, the at least one amino acid is cysteine.

In a preferred embodiment, segments are selected such that they have partial sequence identity or homology with one or more other segments. Suitably, at one or both ends of a respective segment there is comprised at least 4 contiguous amino acids, preferably at least 7 contiguous amino acids, more preferably at least 10 contiguous amino acids, more preferably at least 15 contiguous amino acids and even more preferably at least 20 contiguous amino acids that are identical to, or homologous with, an amino acid sequence contained within one or more other of said segments. Preferably, at the or each end of a respective segment there is comprised less than 500 contiguous amino acids, more preferably less than 200 contiguous amino acids, more preferably less than 100 contiguous amino acids, more preferably less than 50 contiguous amino acids, more preferably less than 40 contiguous amino acids, and even more preferably less than 30 contiguous amino acids that are identical to, or homologous with, an amino acid sequence contained within one or more other of said segments. Such sequence overlap (also referred to elsewhere in the specification as “overlapping fragments” or “overlapping segments”) is preferable to ensure potential epitopes at segment boundaries are not lost and to ensure that epitopes at or near segment boundaries are processed efficiently if placed beside or near amino acids that inhibit processing. Preferably, the segment size is about twice the size of the overlap.

In a preferred embodiment, when segments have partial sequence homology therebetween, the homologous sequences suitably comprise conserved and/or non-conserved amino acid differences.

Conserved or non-conserved differences may correspond to polymorphisms in corresponding parent EBV polypeptides. Polymorphic polypeptides are expressed by various pathogenic organisms and cancers. For example, the polymorphic polypeptides may be expressed by different viral strains or clades or by cancers in different individuals.

Sequence overlap between respective segments is preferable to minimise destruction of any epitope sequences that may result from any shuffling or rearrangement of the segments relative to their existing order in the parent EBV polypeptide. If overlapping segments as described above are employed to form a synthetic polypeptide, it may not be necessary to change the order in which those segments are linked together relative to the order in which corresponding segments are normally present in the parent EBV polypeptide. In this regard, such overlapping segments when linked together in the synthetic polypeptide can adopt a different structure relative to the structure of the parent EBV polypeptide, wherein the different structure does not provide for one or more functions associated with the parent polypeptide. For example, in the case of four segments A-B-C-D each spanning 30 contiguous amino acids of the parent EBV polypeptide and having a 10-amino acid overlapping sequence with one or more adjacent segments, the synthetic polypeptide will have duplicated 10-amino acid sequences bridging segments A-B, B-C and C-D. The presence of these duplicated sequences may be sufficient to render a different structure and to abrogate or alter function relative to the parent EBV polypeptide.

In a preferred embodiment, segment size is about 30 amino acids and sequence overlap at one or both ends of a respective segment is about 15 amino acids. However, it will be understood that other suitable segment sizes and sequence overlap sizes are contemplated by the present invention, which can be readily ascertained by persons of skill in the art.

It is preferable but not necessary to utilise all the segments of the parent EBV polypeptide in the construction of the synthetic polypeptide. Suitably, at least 30%, preferably at least 40%, more preferably at least 50%, even more preferably at least 60%, even more preferably at least 70%, even more preferably at least 80% and still even more preferably at least 90% of the parent EBV polypeptide sequence is used in the construction of the synthetic polypeptide. However, it will be understood that the more sequence information from a parent EBV polypeptide that is utilised to construct the synthetic polypeptide, the greater the population coverage will be of the synthetic polypeptide as an immunogen. Preferably, no sequence information from the parent EBV polypeptide is excluded (e.g., because of an apparent lack of immunological epitopes).

### Preparation of Synthetic Polypeptides

Persons of skill in the art will appreciate that when preparing a synthetic polypeptide against EBV or a cancer caused by, resulting from, or associated with EBV, it may be preferable to use sequence information from a plurality of different polypeptides expressed by EBV or the cancer. Accordingly, in a preferred embodiment, segments from a plurality of different parent EBV polypeptides are linked together to form a synthetic polypeptide according to the invention. It is preferable in this respect to utilize as many parent EBV polypeptides as possible from, or in relation to, a particular source in the construction of the synthetic polypeptide. In particular, it is preferable to utilize EBNA1, LMP1 and LMP2 polypeptides.

Suitably, any hypervariable sequences within the parent EBV polypeptide are excluded from the construction of the synthetic polypeptide.

The synthetic polypeptides of the inventions may be prepared by any suitable procedure known to those of skill in the art. For example, the polypeptide may be synthesised using solution synthesis or solid phase synthesis as described, for example, in Chapter 9 of Atherton and Shephard (1989, Solid Phase Peptide Synthesis: A Practical Approach. IRL Press, Oxford) and in Roberge et al (1995, Science 269: 202). Syntheses may employ, for example, either t-butyloxycarbonyl (t-Boc) or 9-fluorenylmethyloxycarbonyl (Fmoc) chemistries (see Chapter 9.1, of Coligan et al., CURRENT PROTOCOLS IN PROTEIN SCIENCE, John Wiley & Sons, Inc. 1995-1997; Stewart and Young, 1984, Solid Phase Peptide Synthesis, 2nd ed. Pierce Chemical Co., Rockford, Ill.; and Atherton and Shephard, supra).

Alternatively, the polypeptides may be prepared by a procedure including the steps of:

(a) preparing a synthetic construct including a synthetic polynucleotide encoding a synthetic polypeptide wherein said synthetic polynucleotide is operably linked to a regulatory polynucleotide, wherein said synthetic polypeptide comprises a plurality of different segments of a parent polypeptide, wherein said segments are linked together in a different relationship relative to their linkage in the parent EBV polypeptide;

(b) introducing the synthetic construct into a suitable host cell;

(c) culturing the host cell to express the synthetic polypeptide from said synthetic construct; and

(d) isolating the synthetic polypeptide.

Accordingly, the present invention provides synthetic polynucleotides encoding the synthetic polypeptides as described above, as well as synthetic constructs comprising the synthetic polynucleotides operably linked to a regulatory polynucleotide.

The synthetic construct is preferably in the form of an expression vector. For example, the expression vector can be a self-replicating extra-chromosomal vector such as a plasmid, or a vector that integrates into a host genome. Typically, the regulatory polynucleotide may include, but is not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. The regulatory polynucleotide will generally be appropriate for the host cell used for expression. Numerous types of appropriate expression vectors and suitable regulatory polynucleotides are known in the art for a variety of host cells.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the synthetic polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide.

Well known examples of fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG™.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of a fusion polypeptide of the invention, or for isolating cells which express a fusion polypeptide of the invention. Flow cytometric methods such as fluorescence activated cell sorting (FACS) are particularly useful in this latter application. Preferably, the fusion partners also have protease cleavage sites, such as for Factor Xₐ, Thrombin and inteins (protein introns), which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags. Alternatively, a fusion partner may be provided to promote other forms of immunity. For example, the fusion partner may be an antigen-binding molecule that is immuno-interactive with a conformational epitope on a target antigen or to a post-translational modification of a target antigen (e.g., an antigen-binding molecule that is immuno-interactive with a glycosylated target antigen).

The step of introducing the synthetic construct into the host cell may be effected by any suitable method including transfection, and transformation, the choice of which will be dependent on the host cell employed. Such methods are well known to those of skill in the art.

Synthetic polypeptides of the invention may be produced by culturing a host cell transformed with the synthetic construct. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The synthetic polypeptide may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

The amino acids of the synthetic polypeptide can be any non-naturally occurring or any naturally occurring amino acid. Examples of unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methyl-heptanoic acid, t-butylglycine, norleucine, norvaline, phenylglycine, ornithine, sarcosine, 2-thienyl alanine and/or D-isomers of amino acids.

The invention also contemplates modifying the synthetic polypeptides of the invention using ordinary molecular biological techniques so as to alter their resistance to proteolytic degradation or to optimise solubility properties or to render them more suitable as an immunogenic agent.

### Preparation of Synthetic Polynucleotides

According to embodiments of the invention, the disclosed polynucleotides may have the nucleotide sequence as set forth in the sequence listing or display sufficient sequence identity thereto to hybridise to the nucleotide sequence as set forth in the sequence listing. In alternative embodiments, the nucleotide sequence of the polynucleotide may share at least 30%, 40%, 50%, 60%, 70%, 80%, 85%, 90%, 95%, 96%, 97%, 98% or 99% identity with the nucleotide sequence as set forth in the sequence listing.

The present invention contemplates synthetic polynucleotides encoding the synthetic polypeptides as described above. Polynucleotides encoding segments of a parent EBV polypeptide can be produced by any suitable technique. For example, such polynucleotides can be synthesised de novo using readily available machinery. Sequential synthesis of DNA is described, for example, in U.S. Pat. No. 4,293,652. Instead of de novo synthesis, recombinant techniques may be employed including use of restriction endonucleases to cleave a polynucleotide encoding at least a segment of the parent EBV polypeptide and use of ligases to ligate together in frame a plurality of cleaved polynucleotides encoding different segments of the parent polypeptide. Suitable recombinant techniques are described for example in the relevant sections of Ausubel, et al. (supra) and of Sambrook, et al., (supra) which are incorporated herein by reference. Preferably, the synthetic polynucleotide is constructed using splicing by overlapping extension (SOEing) as for example described by Horton et al. (1990, Biotechniques 8(5): 528-535; 1995, Mol Biotechnol. 3(2): 93-99; and 1997, Methods Mol Biol. 67: 141-149). However, it should be noted that the present invention is not dependent on, and not directed to, any one particular technique for constructing the synthetic construct.

Various modifications to the synthetic polynucleotides may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

The invention therefore contemplates a method of producing a synthetic polynucleotide as broadly described above, comprising linking together in the same reading frame at least two nucleic acid sequences encoding different segments of a parent polypeptide to form a synthetic polynucleotide, which encodes a synthetic polypeptide according to the invention. Suitably, nucleic acid sequences encoding at least 10 segments, preferably at least 20 segments, more preferably at least 40 segments and more preferably at least 100 segments of a parent polypeptide are employed to produce the synthetic polynucleotide.

Preferably, the method further comprises selecting segments of the parent EBV polypeptide, reverse translating the selected segments and preparing nucleic acid sequences encoding the selected segments. It is preferred that the method further comprises randomly linking the nucleic acid sequences together to form the synthetic polynucleotide. The nucleic acid sequences may be oligonucleotides or polynucleotides.

Suitably, segments are selected on the basis of size. Additionally, or in the alternative, segments are selected such that they have partial sequence identity or homology (i.e., sequence overlap) with one or more other segments. A number of factors can influence segment size and sequence overlap as mentioned above. In the case of sequence overlap, large amounts of duplicated nucleic acid sequences can sometimes result in sections of nucleic acid being lost during nucleic acid amplification (e.g., polymerase chain reaction, PCR) of such sequences, recombinant plasmid propagation in a bacterial host or during amplification of recombinant viruses containing such sequences. Accordingly, in a preferred embodiment, nucleic acid sequences encoding segments having sequence identity or homology with one or more other encoded segments are not linked together in an arrangement in which the identical or homologous sequences are contiguous. Also, it is preferable that different codons are used to encode a specific amino acid in a duplicated region. In this context, an amino acid of a parent polypeptide sequence is preferably reverse translated to provide a codon which, in the context of adjacent or local sequence elements, has a lower propensity of forming an undesirable sequence (e.g., a duplicated sequence or a palindromic sequence) that is refractory to the execution of a task (e.g., cloning or sequencing). Alternatively, segments may be selected such that they contain a carboxyl terminal leucine residue or such that reverse translated sequences encoding the segments contain restriction enzyme sites for convenient splicing of the reverse translated sequences.

The method optionally further comprises linking a spacer oligonucleotide encoding at least one spacer residue between segment-encoding nucleic acids. Such spacer residue(s) may be advantageous in ensuring that epitopes within the segments are processed and presented efficiently. Preferably, the spacer oligonucleotide encodes 2 to 3 spacer residues. The spacer residue is suitably a neutral amino acid, which is preferably alanine.

Optionally, the method further comprises linking in the same reading frame as other segment-containing nucleic acid sequences at least one variant nucleic acid sequence which encodes a variant segment having a homologous but not identical amino acid sequence relative to other encoded segments. Suitably, the variant segment comprises conserved and/or non-conserved amino acid differences relative to one or more other encoded segments. Such differences may correspond to polymorphisms as discussed above. In a preferred embodiment, degenerate bases are designed or built in to the at least one variant nucleic acid sequence to give rise to all desired homologous sequences.

Preferably, the method further comprises optimising the codon composition of the synthetic polynucleotide such that it is translated efficiently by a host cell. In this regard, it is well known that the translational efficiency of different codons varies between organisms and that such differences in codon usage can be utilised to enhance the level of protein expression in a particular organism. In this regard, reference may be made to Seed et al. (International Application Publication No WO 96/09378) who disclose the replacement of existing codons in a parent EBV polynucleotide with synonymous codons to enhance expression of viral polypeptides in mammalian host cells. This may also have the effect of stabilizing the polynucleotide encoding segments. Preferably, the first or second most frequently used codons are employed for codon optimisation.

Synthetic polynucleotides according to the invention can be operably linked to a regulatory polynucleotide in the form a synthetic construct as for example described above. Synthetic constructs of the invention have utility inter alia as nucleic acid vaccines. The choice of regulatory polynucleotide and synthetic construct will depend on the intended host.

Exemplary expression vectors for expression of a synthetic polypeptide according to the invention include, but are not restricted to, a replication-deficient adenovirus vector based on adenovirus 5 with a fibre protein from adenovirus 35 (Ad5F35). In addition, modified Ankara Vaccinia virus as described, for example, by Allen et al. (2000, J. Immunol. 164(9): 4968-4978), fowlpox virus as for example described by Boyle and Coupar (1988, Virus Res. 10: 343-356) and the herpes simplex amplicons described for example by Fong et al. in U.S. Pat. No. 6,051,428 may also be employed. Alternatively, Epstein-Barr Virus vectors, which are preferably capable of accepting large amounts of DNA or RNA sequence information, can be used.

Preferred promoter sequences that can be utilised for expression of synthetic polypeptides include the P7.5 or PE/L promoters as for example disclosed by Kumar and Boyle. (1990, Virology 179:151-158), CMV and RSV promoters.

The synthetic construct optionally further includes a nucleic acid sequence encoding an immunostimulatory molecule. The immunostimulatory molecule may be fusion partner of the synthetic polypeptide. Alternatively, the immunostimulatory molecule may be translated separately from the synthetic polypeptide. Preferably, the immunostimulatory molecule comprises a general immunostimulatory peptide sequence. For example, the immunostimulatory peptide sequence may comprise a domain of an invasin protein (Inv) from the bacteria Yersinia spp as for example disclosed by Brett et al. (1993, Eur. J. Immunol. 23: 1608-1614).

In an alternate embodiment, the immunostimulatory molecule may comprise an immunostimulatory membrane or soluble molecule, which is suitably a T cell co-stimulatory molecule. Preferably, the T cell co-stimulatory molecule is a B7 molecule or a biologically active fragment thereof, or a variant or derivative of these. The B7 molecule includes, but is not restricted to, B7-1 and B7-2. Preferably, the B7 molecule is B7-1. Alternatively, the T cell co-stimulatory molecule may be an ICAM molecule such as ICAM-1 and ICAM-2.

In another embodiment, the immunostimulatory molecule can be a cytokine, which includes, but is not restricted to, an interleukin, a lymphokine, tumour necrosis factor and an interferon. Alternatively, the immunostimulatory molecule may comprise an immunomodulatory oligonucleotide as for example disclosed by Krieg in U.S. Pat. No. 6,008,200.

Suitably, the size of the synthetic polynucleotide does not exceed the ability of host cells to transcribe, translate or proteolytically process and present epitopes to the immune system. Practitioners in the art will also recognise that the size of the synthetic polynucleotide can impact on the capacity of an expression vector to express the synthetic polynucleotide in a host cell. In this connection, it is known that the efficacy of DNA vaccination reduces with expression vectors greater that 20-kb. In such situations it is preferred that a larger number of smaller synthetic constructs is utilised rather than a single large synthetic construct.

### Compositions and Immunopotentiating Agents

The present invention also contemplates compositions comprising an immunopotentiating agent selected from the group consisting of the synthetic polypeptide, the synthetic polynucleotide and the synthetic construct as described above, together with a pharmaceutically acceptable carrier.

The immunopotentiating agents may be formulated into a composition as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric, maleic, and the like. Salts formed with the free carboxyl groups may also be derived from inorganic basis such as, for example, sodium, potassium, ammonium, calcium, or ferric hydroxides, and such organic basis as isopropylamine, trimethylamine, 2-ethylamino ethanol, histidine, procaine, and the like.

In general, suitable compositions may be prepared according to methods which are known to those of ordinary skill in the art and may include pharmaceutically acceptable diluents, adjuvants and/or excipients. The diluents, adjuvants and excipients must be “acceptable” in terms of being compatible with the other ingredients of the composition, and not deleterious to the recipient thereof.

Examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions. Most preferably, the diluent is saline.

For administration as an injectable solution or suspension, non-toxic parenterally acceptable diluents or carriers can include, Ringer's solution, medium chain triglyceride (MCT), isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

One or more immunopotentiating agents can be used as actives in the preparation of immunopotentiating compositions. Such preparation uses routine methods known to persons skilled in the art. Typically, such compositions are prepared as injectables, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient.

### Routes of Administration

According to the methods of present invention, compounds and compositions may be administered by any suitable route, either systemically, regionally or locally. The particular route of administration to be used in any given circumstance will depend on a number of factors, including the nature of the disease to be treated, the severity and extent of the disease, the required dosage of the particular compounds to be delivered and the potential side-effects of the compounds.

For example, in circumstances where it is required that appropriate concentrations of the desired compounds are delivered directly to the site in the body to be treated, administration may be regional rather than systemic. Regional administration provides the capability of delivering very high local concentrations of the desired compounds to the required site and thus is suitable for achieving the desired therapeutic or preventative effect whilst avoiding exposure of other organs of the body to the compounds and thereby potentially reducing side effects.

By way of example, administration according to embodiments of the invention may be achieved by any standard routes, including intracavitary, intravesical, intramuscular, intraarterial, intravenous, subcutaneous, topical or oral. Intracavitary administration may be intraperitoneal or intrapleural. In particular embodiments, administration may be via intravenous infusion or intraperitoneal administration. Most preferably, administration may be via intravenous infusion.

If desired, devices or compositions containing the immunopotentiating agents suitable for sustained or intermittent release could be, in effect, implanted in the body or topically applied thereto for the relatively slow release of such materials into the body.

Administration of the gene therapy construct to a mammal, preferably a human, may include delivery via direct oral intake, systemic injection, or delivery to selected tissue(s) or cells, or indirectly via delivery to cells isolated from the mammal or a compatible donor. An example of the latter approach would be stem cell therapy, wherein isolated stem cells having potential for growth and differentiation are transfected with the vector comprising the Sox18 nucleic acid. The stem cells are cultured for a period and then transferred to the mammal being treated.

With regard to nucleic acid based compositions, all modes of delivery of such compositions are contemplated by the present invention. Delivery of these compositions to cells or tissues of an animal may be facilitated by microprojectile bombardment, liposome mediated transfection (e.g., lipofectin or lipofectamine), electroporation, calcium phosphate or DEAE-dextran-mediated transfection, for example. In an alternate embodiment, a synthetic construct may be used as a therapeutic or prophylactic composition in the form of a “naked DNA” composition as is known in the art. A discussion of suitable delivery methods may be found in Chapter 9 of CURRENT PROTOCOLS IN MOLECULAR BIOLOGY (Eds. Ausubel et al.; John Wiley & Sons Inc., 1997 Edition) or on the Internet site DNAvaccine.com. The compositions may be administered by intradermal (e.g., using Panjet™ delivery) or intramuscular routes.

The step of introducing the synthetic polynucleotide into a target cell will differ depending on the intended use and species, and can involve one or more of non-viral and viral vectors, cationic liposomes, retroviruses, and adenoviruses such as, for example, described in Mulligan, R. C., (1993 Science 260 926-932) which is hereby incorporated by reference. Such methods can include, for example:

A. Local application of the synthetic polynucleotide by injection (Wolff et al., 1990, Science 247 1465-1468, which is hereby incorporated by reference), surgical implantation, instillation or any other means. This method can also be used in combination with local application by injection, surgical implantation, instillation or any other means, of cells responsive to the protein encoded by the synthetic polynucleotide so as to increase the effectiveness of that treatment. This method can also be used in combination with local application by injection, surgical implantation, instillation or any other means, of another factor or factors required for the activity of said protein.

B. General systemic delivery by injection of DNA, (Calabretta et al., 1993, Cancer Treat. Rev. 19 169-179, which is incorporated herein by reference), or RNA, alone or in combination with liposomes (Zhu et al., 1993, Science 261 209-212, which is incorporated herein by reference), viral capsids or nanoparticles (Bertling et al., 1991, Biotech. Appl. Biochem. 13 390-405, which is incorporated herein by reference) or any other mediator of delivery. Improved targeting might be achieved by linking the synthetic polynucleotide to a targeting molecule (the so-called “magic bullet” approach employing, for example, an antibody), or by local application by injection, surgical implantation or any other means, of another factor or factors required for the activity of the protein encoding said synthetic polynucleotide, or of cells responsive to said protein.

C. Injection or implantation or delivery by any means, of cells that have been modified ex vivo by transfection (for example, in the presence of calcium phosphate: Chen et al., 1987, Mole. Cell Biochem. 7 2745-2752, or of cationic lipids and polyamines: Rose et al., 1991, BioTech. 10 520-525, which articles are incorporated herein by reference), infection, injection, electroporation (Shigekawa et al., 1988, BioTech. 6 742-751, which is incorporated herein by reference) or any other way so as to increase the expression of said synthetic polynucleotide in those cells. The modification can be mediated by plasmid, bacteriophage, cosmid, viral (such as adenoviral or retroviral; Mulligan, 1993, Science 260 926-932; Miller, 1992, Nature 357 455-460; Salmons et al., 1993, Hum. Gen. Ther. 4 129-141, which articles are incorporated herein by reference) or other vectors, or other agents of modification such as liposomes (Zhu et al., 1993, Science 261 209-212, which is incorporated herein by reference), viral capsids or nanoparticles (Bertling et al., 1991, Biotech. Appl. Biochem. 13 390-405, which is incorporated herein by reference), or any other mediator of modification. The use of cells as a delivery vehicle for genes or gene products has been described by Barr et al., 1991, Science 254 1507-1512 and by Dhawan et al., 1991, Science 254 1509-1512, which articles are incorporated herein by reference. Treated cells can be delivered in combination with any nutrient, growth factor, matrix or other agent that will promote their survival in the treated subject.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

### Dosages

The effective dose level of the administered compound for any particular subject will depend upon a variety of factors including: the type of disease being treated and the stage of the disease; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis.

In terms of weight, a therapeutically effective dosage of a composition for administration to a patient is expected to be in the range of about 0.01 mg to about 150 mg per kg body weight per 24 hours; typically, about 0.1 mg to about 150 mg per kg body weight per 24 hours; about 0.1 mg to about 100 mg per kg body weight per 24 hours; about 0.5 mg to about 100 mg per kg body weight per 24 hours; or about 1.0 mg to about 100 mg per kg body weight per 24 hours. More typically, an effective dose range is expected to be in the range of about 5 mg to about 50 mg per kg body weight per 24 hours.

Alternatively, an effective dosage may be up to about 5000 mg/m². Generally, an effective dosage is expected to be in the range of about 10 to about 5000 mg/m², typically about 10 to about 2500 mg/m², about 25 to about 2000 mg/m², about 50 to about 1500 mg/m², about 50 to about 1000 mg/m², or about 75 to about 600 mg/m².

Further, it will be apparent to one of ordinary skill in the art that the optimal quantity and spacing of individual dosages will be determined by the nature and extent of the condition being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per unit time, can be ascertained by those skilled in the art using conventional course of treatment determination tests.

### Methods of Treatment

Also encapsulated by the present invention are methods for modulating an immune response, which response is directed against an EBV-associated disease, comprising administering to a patient in need of such treatment an effective amount of an immunopotentiating agent selected from the group consisting of the vaccines, the synthetic polypeptides, the synthetic polynucleotides, the synthetic constructs, or the compositions as described above.

Moreover, the present invention also provides methods for treatment and/or prophylaxis of an EBV-associated disease, comprising administering to a patient in need of such treatment an effective amount of an immunopotentiating agent selected from the group consisting of the vaccines, the synthetic polypeptides, the synthetic polynucleotides, the synthetic constructs, or the compositions as described above.

In a preferred embodiment, the immunopotentiating composition of the invention is suitable for treatment of, or prophylaxis against, a cancer. Cancers which could be suitably treated in accordance with the practices of this invention include nasopharyngeal carcinoma, Hodgkin's lymphoma and post-transplant lymphoproliferative disease.

In an additional or alternative embodiment, the immunopotentiating composition is suitable for treatment of, or prophylaxis against, a viral infection. Viral infections contemplated by the present invention encompass infections caused by Epstein-Barr virus.

### Assessment of Immunisation Efficacy

The effectiveness of the immunisation may be assessed using any suitable technique. For example, CTL lysis assays may be employed using stimulated splenocytes or peripheral blood mononuclear cells (PBMC) on peptide coated or recombinant virus infected cells using .sup.51Cr labelled target cells. Such assays can be performed using for example primate, mouse or human cells (Allen et al., 2000, J. Immunol. 164(9): 4968-4978 also Woodberry et al., infra). Alternatively, the efficacy of the immunisation may be monitored using one or more techniques including, but not limited to, HLA class I Tetramer staining of both fresh and stimulated PBMCs (see for example Allen et al., supra), proliferation assays (Allen et al., supra), Elispot™ Assays and intracellular INF-gamma staining (Allen et al., supra), ELISA Assays for linear B cell responses; and Western blots of cell sample expressing the synthetic polynucleotides.

### Design and Production of Synthetic Polypeptides

The design or construction of a synthetic polypeptide sequence or a synthetic polynucleotide sequence according to the invention is suitably facilitated with the assistance of a computer programmed with software, which inter alia fragments a parent EBV sequence into fragments, and which links those fragments together in a different relationship relative to their linkage in the parent EBV sequence. The ready use of a parent EBV sequence for the construction of a desired synthetic molecule according to the invention requires that it be stored in a computer-readable format. Thus, in accordance with the present invention, sequence data relating to a parent molecule (e.g. a parent polypeptide) is stored in a machine-readable storage medium, which is capable of processing the data to fragment the sequence of the parent molecule into fragments and to link together the fragments in a different relationship relative to their linkage in the parent molecule.

Therefore, the disclosure herein also relates to a machine-readable data storage medium, comprising a data storage material encoded with machine readable data which, when used by a machine programmed with instructions for using said data, fragments a parent sequence into fragments, and links those fragments together in a different relationship relative to their linkage in the parent sequence. In a preferred embodiment of this type, a machine-readable data storage medium is provided that is capable of reverse translating the sequence of a respective fragment to provide a nucleic acid sequence encoding the fragment and to link together in the same reading frame each of the nucleic acid sequences to provide a polynucleotide sequence that codes for a polypeptide sequence in which said fragments are linked together in a different relationship relative to their linkage in a parent polypeptide sequence.

In another embodiment, the disclosure encompasses a computer for designing the sequence of a synthetic polypeptide and/or a synthetic polynucleotide of the invention, wherein the computer comprises wherein said computer comprises: (a) a machine readable data storage medium comprising a data storage material encoded with machine readable data, wherein said machine readable data comprises the sequence of a parent polypeptide; (b) a working memory for storing instructions for processing said machine-readable data; (c) a central-processing unit coupled to said working memory and to said machine-readable data storage medium, for processing said machine-readable data into said synthetic polypeptide sequence and/or said synthetic polynucleotide; and (d) an output hardware coupled to said central processing unit, for receiving said synthetic polypeptide sequence and/or said synthetic polynucleotide.

In yet another embodiment, the disclosure contemplates a computer program product for designing the sequence of a synthetic polynucleotide of the invention, comprising code that receives as input the sequence of a parent polypeptide, code that fragments the sequence of the parent polypeptide into fragments, code that reverse translates the sequence of a respective fragment to provide a nucleic acid sequence encoding the fragment, code that links together in the same reading frame each said nucleic acid sequence to provide a polynucleotide sequence that codes for a polypeptide sequence in which said fragments are linked together in a different relationship relative to their linkage in the parent polypeptide sequence, and a computer readable medium that stores the codes.

Accordingly, the disclosure relates to a computer program product for designing the sequence of a synthetic polypeptide, comprising:

(a) code that receives as input the sequence of at least one parent EBV polypeptide;

(b) code that fragments the sequence of a respective parent EBV polypeptide into fragments;

(c) code that links together said fragments in a different relationship relative to their linkage in said parent EBV polypeptide sequence; and

(d) a computer readable medium that stores the codes.

The disclosure herein further relates to a computer program product for designing the sequence of a synthetic polynucleotide, comprising:

(a) code that receives as input the sequence of at least one parent EBV polypeptide;

(b) code that fragments the sequence of a respective parent EBV polypeptide into fragments;

(c) code that reverse translates the sequence of a respective fragment to provide a nucleic acid sequence encoding said fragment;

(d) code that links together in the same reading frame each said nucleic acid sequence to provide a polynucleotide sequence that codes for a polypeptide sequence in which said fragments are linked together in a different relationship relative to their linkage in the at least one parent EBV polypeptide sequence; and

(e) a computer readable medium that stores the codes.

The disclosure herein also relates to a computer for designing the sequence of a synthetic polypeptide, wherein said computer comprises:

(a) a machine-readable data storage medium comprising a data storage material encoded with machine-readable data, wherein said machine-readable data comprise the sequence of at least one parent EBV polypeptide;

(b) a working memory for storing instructions for processing said machine-readable data;

(c) a central-processing unit coupled to said working memory and to said machine-readable data storage medium, for processing said machine readable data to provide said synthetic polypeptide sequence; and

(d) an output hardware coupled to said central processing unit, for receiving said synthetic polypeptide sequence.

The processing of said machine readable data may comprise fragmenting the sequence of a respective parent EBV polypeptide into fragments and linking together said fragments in a different relationship relative to their linkage in the sequence of said parent EBV polypeptide.

The disclosure additionally relates to a computer for designing the sequence of a synthetic polynucleotide, wherein said computer comprises:

(a) a machine-readable data storage medium comprising a data storage material encoded with machine-readable data, wherein said machine-readable data comprise the sequence of at least one parent EBV polypeptide;

(b) a working memory for storing instructions for processing said machine-readable data;

(c) a central-processing unit coupled to said working memory and to said machine-readable data storage medium, for processing said machine readable data to provide said synthetic polynucleotide sequence; and

(d) an output hardware coupled to said central processing unit, for receiving said synthetic polynucleotide sequence.

The processing of said machine readable data may comprise fragmenting the sequence of a respective parent EBV polypeptide into fragments, reverse translating the sequence of a respective fragment to provide a nucleic acid sequence encoding said fragment and linking together in the same reading frame each said nucleic acid sequence to provide a polynucleotide sequence that codes for a polypeptide sequence in which said fragments are linked together in a different relationship relative to their linkage in the at least one parent EBV polypeptide sequence.

The present invention will now be further described in greater detail by reference to the following specific examples, which should not be construed as in any way limiting the scope of the invention.

### EXAMPLES

### Example 1

### General Methods

### 1.1 Construction of an NPC SAVINE

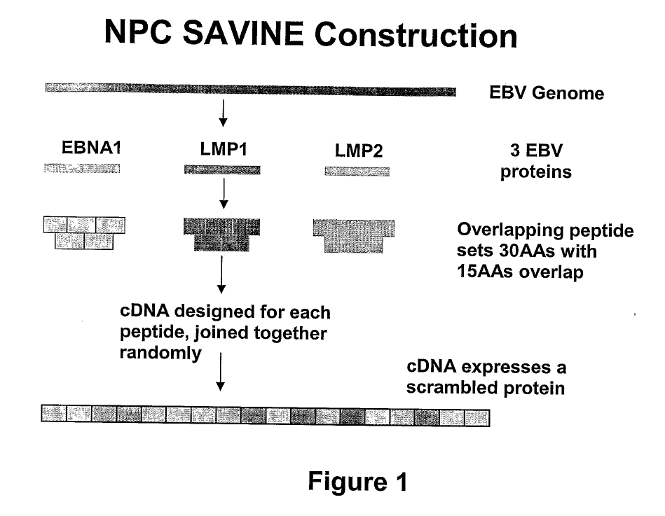

DNA sequences encoding the EBNA1, LMP1 and LMP2 proteins were constructed using sequence-specific overlapping oligonucleotides varying in length from 20 to 100 bp (FIG. 1). Sequences were joined together by stepwise asymmetric PCR to create subcassettes. These subcassettes were joined together using restriction digestion and PCR to develop the final NPC SAVINE construct of 6.8 kb. This construct was then cloned into the replication deficient adenovirus vector Ad5F35. The recombinant adenovirus expressing SAVINE construct (AdSAVINE) was obtained by transfecting into HEK293 cells. This SAVINE construct was also inserted into vaccinia and fowl pox virus delivery vectors (see Thomson S. A., Jaramillo A. B., Shoobridge M., Dunstan K. J., Everett B., Ranasinghe C., Kent S. J., Gao K., Medveckzy C. J., French R. A., Ramshaw I. A. Development Of A Synthetic Consensus Sequence Scrambled Antigen HIV-1 Vaccine Designed for Global Use (2005) Vaccine, 23(38) 4647-57).

### 1.2 Establishment and Maintenance of Cell Lines

EBV-transformed lymphoblastoid cell lines (LCLs) were established from seropositive donors by exogenous virus transformation of peripheral B cells using the B95.8 virus isolate. These cell lines were routinely maintained in RPMI 1640 (Gibco Invitrogen Corp., Carlsbad, Calif.) supplemented with 2 mM L-glutamine, 100 IU/ml penicillin and 100 μg/ml streptomycin plus 10% foetal calf serum (FCS) (referred to as growth medium). In addition, the HEK 293 cell line was maintained in DMEM containing 10% FCS.

### 1.3 Synthesis of Peptides

Peptides, synthesized by the Merrifield solid phase method, were purchased from Chiron Mimotopes (Melbourne, Australia), dissolved in dimethyl sulphoxide, and diluted in serum-free RPMI 1640 medium for use in standard CTL assays. Purity of these peptides were tested by mass spectrometery and showed >90% purity.

1.4 Expansion of LMP-Specific CTL from Human Healthy EBV Donors

Peripheral blood cells from EBV seropositive HLA A2 healthy individuals were activated with the LMP polyepitope formulation. Briefly, 2×10⁶ PBMC were co-cultured in a 24-well plate with autologous PBMC infected with recombinant adenovirus expressing LMP polyepitope (MOI: 50:1) at a responder to stimulator ratio of 50:1. Three days after, growth medium was supplemented with rhIL-2 (20 U/mL). These cultures were restimulated at weekly intervals with autologous LCL infected with recombinant adenovirus expressing LMP polyepitope and supplemented with rhIL-2. For LCL stimulation, 2×10⁶ PBMC were co-cultured with autologous LCLs (irradiated, 8000 rads) at a responder to stimulator ratio of 30:1 and LMP-specific T-cell reactivity was assessed by ELISPOT assay and in vitro cytotoxicity assay.

### 1.5 In Vitro Cytotoxicity Assay and ELISPOT Assay

On day 6 after 3 rounds of in vitro stimulations, CTL activity was measured using ELISPOT and ⁵¹Cr-release assay. For the ELISPOT assay, expanded CTL were incubated in triplicate with relevant peptides (10⁻⁵M) for about 18 h at 37° C. in 96-well mixed cellulose ester membrane plates (Millipore, Bedford, USA) precoated with anti-mouse IFN-γ mAb (Mabtech AB, Nacka, Sweden). (Anti-human IFN-γ mAb and biotinylated anti-human IFN-γ-mAb were used to measure expanded human CTL). After incubation, the plates were extensively washed with PBS containing 0.5% Tween 20 and incubated with a secondary biotinylated anti-mouse IFN-γ-mAb, followed by the addition of streptavidin-alkaline phosphatase. Individual IFN-γ-producing cells were detected as purple spots after reaction with 5-bromo-4-chloro-3-indolyl phosphate and nitro blue tetrazolium. Spots were counted automatically using image analysis software. CTL precursor frequencies for each peptide were calculated as spot-forming cells (SFC) per 10⁶ cultured cells. The number of IFN-γ-secreting T cells was calculated by subtracting the negative control (CTL cultures with irrelevant peptide).

For the in vitro cytotoxicity assay, HLA-A2 restricted human PHA blasts pulsed with the relevant peptide were used as target cells. The percent of specific lysis was calculated as:

$$\frac{100 \times ( experimental release \text{-} spontaneous release )}{( maximum release \text{-} spontaneous release )}$$

### 1.6 Mice

Balb/c nude mice and HLA A2/Kb mice (a kind gift from Dr L. Sherman, Scripps Research Institute, CA) were purchased from the Animal Resource Centre (ARC), WA, Australia. HLA A2/K^{b} transgenic mice express chimeric human (α 1 and α 2 HLA A2 domains) and murine (α 3, transmembrane and cytoplasmic H-2/K^{b} domains) class I molecules. Female HLA A2/K^{b} and nude mice between 6-8 weeks of age were used for all experiments. All experiments were performed under protocols approved by the institute ethics committee.

### 1.7 Tumour Model

Immunodeficient nude mice were subcutaneously implanted in the dorsal side of the neck with human NPC allografts (called C17, kindly provided by Dr. Pierre Busson, Gustav Roussey, Paris) of 2 mm³. C17 was originally derived from metastatic tissue of an NPC patient (HLA type of tumour A2, B41, B45).

1.8 Immunisation of HLA A2/K^{b} Transgenic Mice with SAVINE

HLA-A2/Kb transgenic mice (n=5) were immunised subcutaneously (s.c.) with Ad SAVINE (10⁹ PFU). Two weeks later, these mice were again injected with either Vaccinia-SAVINE (10⁷ PFU) or Fowl pox SAVINE (2×10⁷ PFU).

1.9 In Vitro Expansion of SAVINE-Specific CTL from Spleens of Immunised HLA-A2/Kb Mice

After 3 weeks of immunisation, single cell suspensions of spleen were prepared by pressing the tissue through nylon membrane followed by lysis of RBCs using ACK lysis buffer. Cells were plated at 4×10⁶/well in 24-well plates in RPMI medium containing 10% FBS, 100 u/ml penicillin, 100 ug/ml streptomycin, 2 mM L-glutamine, and 50 uM β-mercaptoethanol (RPMI 1640 complete medium) with 20 U/ml human IL-2. The spleen cells were stimulated using autologous irradiated (2000 rads) splenocytes sensitised with relevant peptides (10⁻⁵M for 1 h at 37° C.) at a responder to stimulator ratio of 4:1. These cultures were restimulated at weekly intervals using allogeneic splenocytes coated with relevant peptides.

### 1.10 Adoptive Transfer

Immunodeficient nude mice were inoculated with human NPC allografts and when the tumour size was approximately 0.2 cm³ in size (14 days after tumour inoculation), each group of tumour-bearing nude mice (n=6 mice/group) was adoptively transferred with either 5×10⁶ Ad (primed)-VV (boosted) SAVINE-specific T cells or 5×10⁶ Ad-FPV SAVINE-specific T cells. Another group of nude mice was injected with 5×10⁶ Ad-FPV SAVINE-CTL and treated with human IL-15 (5 μg) intraperitoneal (i.p.) injection 1, 2 and 3 days after each adoptive transfer. Control groups included were mice injected with 5×10⁶ LMP polyepitope-specific CTL, cytomegalovirus polyepitope (CMV)-specific CTL, CD8 depleted Ad-FPV SAVINE-CTL or untreated. The therapeutic efficacy of SAVINE-specific T cells was assessed by regular monitoring of tumour regression and mice showing a tumour size of >1.0 cm³ in size were sacrificed.

### Example 2

### DNA Sequence Encoding SAVINE Protein

The scrambled DNA sequence encoding the SAVINE protein is disclosed as SEQ ID NO:1. The protein encoded by SEQ ID NO:1 consists of randomised overlapping amino sequences from EBNA1, LMP2 and LMP1. The encoded peptide sequences are 30 amino acids drawn from these proteins overlapping by 15 amino acids. This SAVINE protein has been inserted into Ad5/F35, vaccinia virus and fowlpox virus vectors.

### Example 3

### The Defined Epitopes within the SAVINE Protein Efficiently Process and Present to EBNA1, LMP1 and LMP2 T Cells

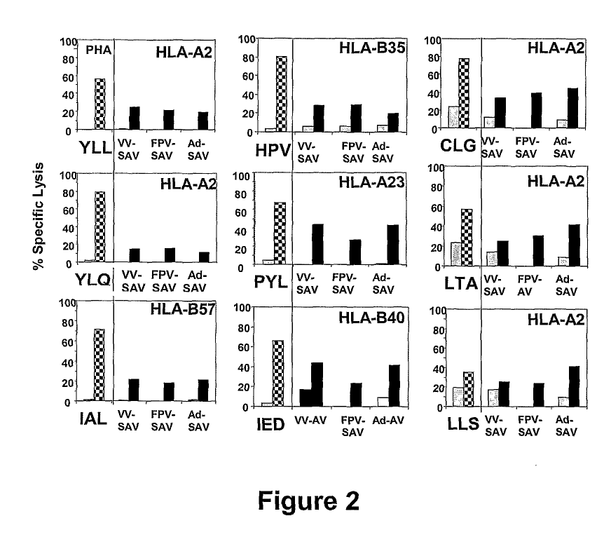

HLA-matched fibroblasts infected with either vaccinia, fowlpox or adenovirus expressing the SAVINE protein showed cytolytic activity against EBNA1, LMP1 and LMP2 peptide-specific CTL whereas the fibroblasts infected with vaccinia TK-, empty adenovirus or uninfected fibroblasts were not lysed (FIG. 2).

FIG. 2 demonstrates that the defined epitope-specific CTL polyclonal lines or CTL clones within EBNA1 (HPV, HLA-B35 restricted), LMP1 (YLL and YLQ, HLA A2-restricted; IAL, HLA B35-restricted) and LMP2 (CLG, LTA and LLS, HLA A2-restricted; PYL, HLA-A23-restricted; IED, HLA-B40-restricted) antigens were generated from four EBV seropositive healthy donors. The specificity of these CTL was tested against the defined epitope-loaded PHA blasts in a cytolytic assay. Subsequently, to find out whether the defined epitopes within EBNA1, LMP1 and LMP2 antigens were endogenously processed, HLA-matched fibroblasts were first infected with vaccinia, fowl pox or adenovirus vectors expressing SAVINE construct (MOI, 10:1). The target fibroblasts infected with vaccinia TK-, empty adenovirus or uninfected fibroblasts were used as controls. These targets were then tested for the cytolytic activity against EBNA1, LMP1 and LMP2 epitope-specific CTL polyclonal lines or CTL clones generated from EBV seropositive healthy donors in a Chromium release assay. An Effector:Target ratio of 10:1 is used in these assays. HLA-matched fibroblasts infected with either vaccinia, fowl pox or adenovirus vectors expressing SAVINE construct showed cytolytic activity, whereas fibroblasts infected with control vectors were not lysed.

These results demonstrate that the defined epitopes in the SAVINE construct are processed and presented to the targets cells very efficiently.

### Example 4

### Activation of SAVINE-Specific CTL from EBV Immune Healthy Donors

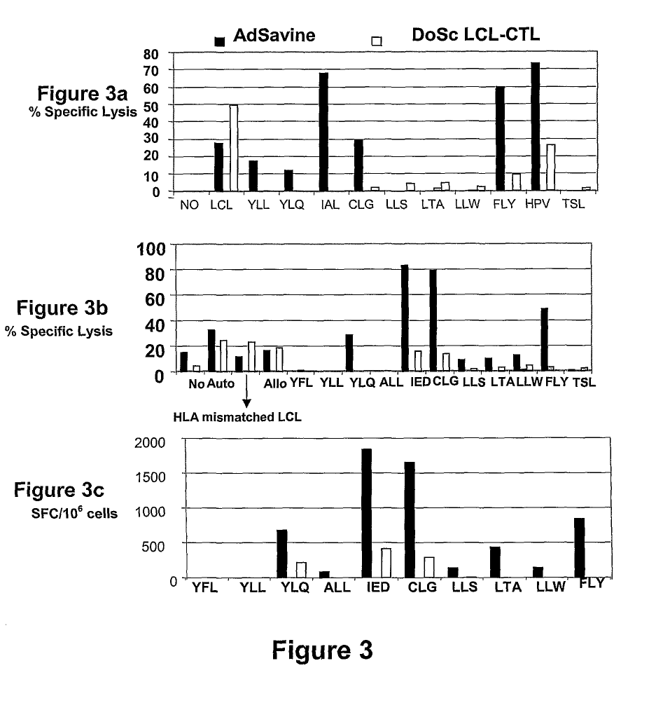

PBMCs from healthy human EBV carriers (ScBu and DoSc) were stimulated with autologous PBMCs infected (responder to stimulator ratio of 2:1) with either AdSAVINE, AdPoly or autologous LCL (30:1) (FIGS. 3(a) and (b)). All cultures were restimulated at weekly intervals using γ-irradiated autologous LCLs infected as described. Three days after 3 restimulations the cultured cells were used as effectors in a Chromium release assay against peptide-sensitized autologous PHA blasts. The cultured cells were also tested by ELISPOT and the results are expressed as spot forming cells (SFC) per 10⁶ CTL (FIG. 3(c)).

Stimulation of PBMC from healthy donors with either adenovirus SAVINE of autologous LCLs, with effector function testing using chromium release assays and by ELISPOT assays (FIG. 3(a), (b) and (c)) therefore shows that the SAVINE-activated CTL shows specific lysis that is higher than the LCL-activated CTL.

### Example 5

### Mapping New Responses with the SAVINE Construct

The amino acid sequences of full length LMP1 antigen were derived from both Asian EBV strain, CAO (32 peptides of 17 mer in length overlapping by 8 residues) and Caucasian prototype 1 EBV strain, B95.8 (42 peptides of 17 mer in length overlapping by 8 residues). The amino acid sequences of full length LMP2 (49 peptides of 20 mer in length overlapping by 10 residues) and EBNA1 (69 peptides of 15 mer in length overlapping by 10 residues) antigens were derived from Caucasian prototype 1 EBV strain, B95.8. Adenovirus-SAVINE and LCL-activated CTL generated from four EBV seropositive healthy donors were tested for the secretion of IFN-γ after stimulation with overlapping peptides. Specific T cell reactivity to defined CD8⁺ as well as CD4⁺ T cell epitopes were observed. In addition to reactivity against already defined peptides, four of these new peptide pool sequences (2 each from LMP1 and LMP2) showed reactivity by both SAVINE and LCL-activated CTL and four of these new peptide pool sequences (1 each from CAO LMP1, B95.8 LMP1 LMP2 and EBNA1) showed reactivity by SAVINE activated CTL.

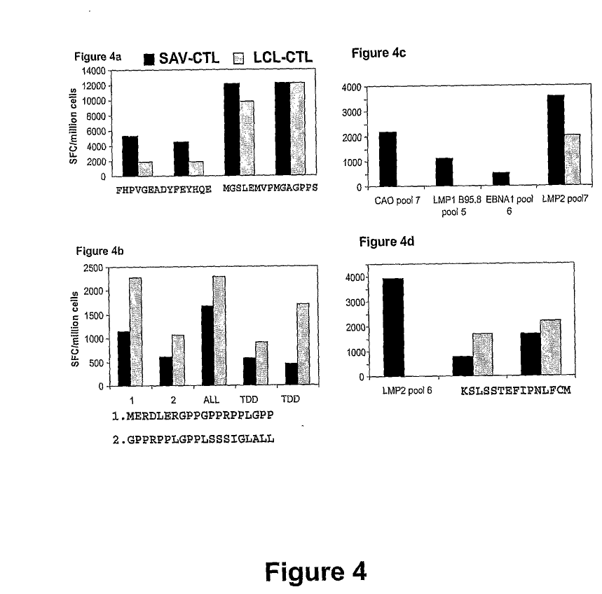

Screening of the SAVINE-activated CTL with a panel of peptides from EBNA1, LMP1 and LMP2 (FIGS. 4(a), (b), (c) and (d)) therefore shows that the SAVINE construct activated already defined CTL epitopes from each of the three proteins. In addition, the SAVINE activated reactivity to 4 new pooled peptide sequences.

### Example 6

### The Ad5/F35 SAVINE Construct can Prime a CTL Response in Mice which can be Boosted with Either Vaccinia SAVINE or Fowlpox SAVINE

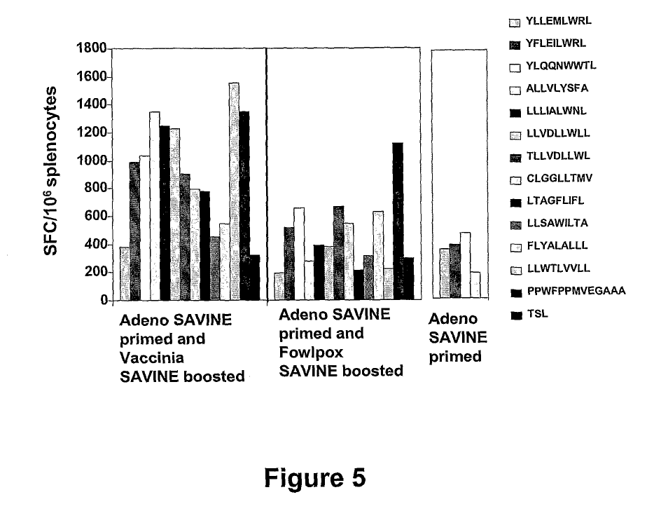

Two groups of HLA-A2/Kb transgenic mice (n=5) were immunised s.c. with Ad SAVINE (10⁹ PFU) and two weeks later, these mice were again injected with either Vaccinia-SAVINE (10⁷ PFU) or Fowl pox SAVINE (2×10⁷ PFU). Two weeks later, the spleen cells were harvested and CTL response was assessed by ELISPOT assays and the results are expressed as mean+SE of spot-forming cells (SFC) per 10⁶ splenocytes (FIG. 5).

FIG. 5 therefore demonstrates that HLA A2 Kb mice immunised with the Ad5/F35 SAVINE prime a specific CTL response and that this response can be measured ex vivo in spleen cells by ELISPOT assay. This priming CTL response can be boosted following immunisation with either vaccinia SAVINE or fowlpox SAVINE.

### Example 7

### Therapeutic Efficacy of In Vitro Expanded SAVINE CTL Cause Regression of Human NPC

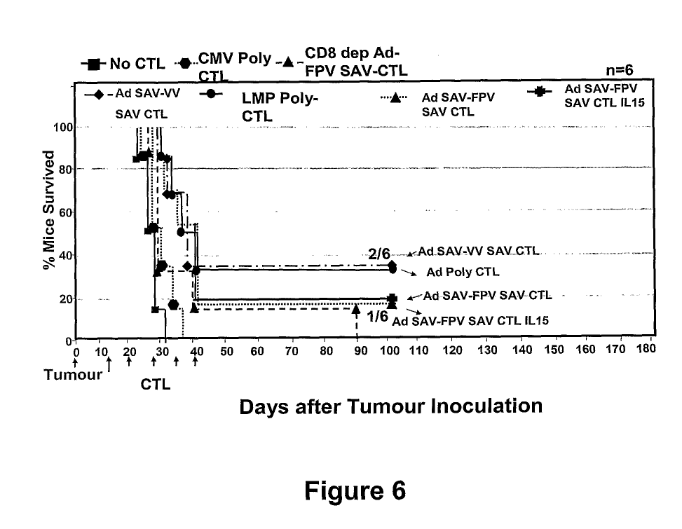

Immunodeficient nude mice were inoculated with human NPC allografts and when the tumour size was approximately 0.2 cm³ in size (14 days after tumour inoculation), each group of tumour-bearing nude mice (n=6 mice/group) was adoptively transferred with either 5×10⁶ Ad (primed)-VV (boosted) SAVINE-specific T cells or 5×10⁶ Ad-FPV SAVINE-specific T cells. Another group of nude mice was injected with 5×10⁶ Ad-FPV SAVINE-CTL and treated with human IL-15 (5 μg) injection i.p. 1, 2 and 3 days after each adoptive transfer. Control groups included were mice injected with 5×10⁶ LMP polyepitope-specific CTL, cytomegalovirus polyepitope (CMV)-specific CTL, CD8 depleted Ad-FPV SAVINE-CTL or untreated. The therapeutic efficacy of SAVINE-specific T cells was assessed by regular monitoring of tumour regression and mice showing a tumour size of >1.0 cm³ in size were sacrificed. Untreated mice, mice that received CMV T cells or CD8 depleted Ad-FPV SAVINE-CTL did not result in inhibition of tumour growth and the tumours in these mice reached 1.0 cm³ by about 12-24 days after the first T cell transfer. Mice receiving CD8 depleted LMP-CTL were sacrificed by about 12-78 days after first CTL transfer. After 90 days, 1/6 mice receiving either Ad-FPV SAVINE-CTL alone or mice receiving Ad-FPV SAVINE-CTL as well as IL15 sustained regression and the regression in 2/6 mice sustained in mice that received Ad-VV SAVINE-CTL (FIG. 6).

FIG. 6 therefore demonstrates that SAVINE CTL from mice prime boosted as in FIG. 5 and subsequently expanded in vitro using defined epitope CTL peptides can protect nude mice in which human NPC cells are growing.

1. A vaccine for the treatment or prevention of an EBV-associated disease in a subject, wherein said vaccine comprises a synthetic polypeptide comprising a plurality of different segments of at least one parent EBV polypeptide, and wherein the segments are linked together in a different relationship relative to their linkage in the at least one parent EBV polypeptide, and wherein at least one of said parent EBV polypeptides is selected from the group including EBNA1, LMPI and LMP2 and wherein repetitive sequences of said peptides are substantially eliminated.

2. The vaccine of claim 1, wherein the EBV-associated disease is cancer.

3. The vaccine of claim 2, wherein the cancer is selected from the group including nasopharyngeal carcinoma (NPC), Hodgkin's lymphoma (HL) and post-transplant lymphoproliferative disease (PTLD).

4. The vaccine of claim 1, wherein the synthetic polypeptide consists essentially of different segments of a single parent EBV polypeptide.

5. The vaccine of claim 1, wherein the synthetic polypeptide consists essentially of different segments of a plurality of different parent EBV polypeptides.

6. The vaccine of claim 1, wherein at least one of said segments comprises partial sequence identity or homology to one or more other said segments.

7. The vaccine of claim 6, wherein the sequence identity or homology is contained at one or both ends of said at least one segment.

8. A synthetic polypeptide, wherein said polypeptide comprises a plurality of different segments of at least one parent EBV polypeptide, and wherein the segments are linked together in a different relationship relative to their linkage in the at least one parent EBV polypeptide, and wherein at least one of said parent EBV polypeptides is selected from the group including EBNA1, LMP1 and LMP2 and wherein repetitive sequences of said peptides are substantially eliminated.

9. A synthetic polynucleotide encoding the synthetic polypeptide of claim 8.

10. The synthetic polynucleotide of claim 9, wherein said synthetic polynucleotide comprises the sequence as set forth at SEQ ID NO: 1.

11. A synthetic construct comprising the polynucleotide of claim 9 operably linked to a regulatory polynucleotide.

12. A method for producing the synthetic polynucleotide of claim 9, comprising linking together in the same reading frame a plurality of nucleic acid sequences encoding different segments of at least one parent EBV polypeptide to form a synthetic polynucleotide whose sequence encodes said segments linked together in a different relationship relative to their linkage in the at least one parent EBV polypeptide.

13. The method of claim 12, further comprising fragmenting the sequence of a respective parent EBV polypeptide into fragments and linking said fragments together in a different relationship relative to their linkage in said parent EBV polypeptide sequence.

14. The method of claim 13, wherein said fragments are randomly linked together.

15. The method of claim 12, further comprising reverse translating the sequence of a respective parent EBV polypeptide or a segment thereof to provide a nucleic acid sequence encoding said parent EBV polypeptide or said segment.

16. The method of claim 15, wherein an amino acid of said parent EBV polypeptide sequence is reverse translated to provide a codon which has higher translational efficiency than other synonymous codons in a cell of interest.

17. The method of claim 16, wherein the amino acid of said parent EBV polypeptide sequence is reverse translated to provide a codon which, in the context of adjacent or local sequence elements, has a lower propensity of fanning an undesirable sequence that is refractory to the execution of a task.

18. The method of claim 17, wherein the undesirable sequence is a palindromic sequence or a duplicated sequence.

19. The method of claim 17, wherein the task is cloning, sequencing, enhancing the stability of the polynucleotide or enhancing in vivo translation.

20. A composition comprising an immunopotentiating agent selected from the group consisting of: the synthetic polypeptide of claim 8, and the synthetic polynucleotide of claim 9, together with a pharmaceutically acceptable carrier.

21. The composition of claim 20, further comprising an adjuvant.

22. A method for modulating an immune response, which response is directed against an EBV-associated disease, comprising administering to a patient in need of such treatment an effective amount of an immunopotentiating agent selected from the group consisting of: the synthetic polypeptide of claim 8, and the synthetic polynucleotide of claim 9.

23. A method for treatment and/or prophylaxis of an EBV-associated disease, comprising administering to a patient in need of such treatment an effective amount of an immunopotentiating agent selected from the group consisting of: the synthetic polypeptide of claim 8, and the synthetic polynucleotide of claim 9.

24.-26. (canceled)


SEQUENCE LISTING (1 sequence)

SEQ ID NO: 1 - DNA, 6875 bp, Artificial Sequence
  Synthetic polynucleotide
ggcggatcctctagaccaccatgggcgctaccctctggcgactgctcgcctttatcctcgcctttttcctcgccattatcctcctgattatcgctctgtatctgcaacagaatgccctcatgctcatcattatcattctgattatctttatctttaggagagacctcctgtgtcccctcggcggactgggactgctcctgctcggccctcacgatcccctcccccataaccctagcgatagcgctggcaatgacggaggccctcccaatctgacagaggaagtggctaacaaaatccctaacctcttctgtatgctcctgctcatcgtcgccggaatcctcttcattctggctatcctcaccgaatggggaagcggaaacagaaggcctcccctcggccctcccctcagctccagcatcggcctcgccctcctgctcctgctcctggctctgctcttctggctgtatatcgtctaccaacccctcggcacacaggatcagtccctgtatctgggactgcaacacgatggcaatgacggactgcctccccctccctatagccctggcggagacagaggccctccctccatgacagacggaggcggaggcgatccccatctgcctaccctcctgctcggcacaagcggaagcggagtggctggcgctgtgtggctgacagtgatgagcaataccctcctgtccgcctggattctcaccgctggctttctgattttcctcatcggaaagcctgcccctacctgtaacattagggtcaccgtctgctccttcgatgacggagtggatctgcctccctggttccctcccatggtggaagccgctgagaaaaggcctaggtccccctccagccaaagctccagctccggctccccccctaggagaccccctcccggaaggagaccctttagcatgaaccctgtgtgtctgcctgtgattgtggctccctatctgttttggctcgccgctatcgctgcctcctgctttaccgctagcgtctactgtctgacactggaaagcgaagagagaccccctaccccttacgctgcctggagaaggctcaccgtctgcggaggcattatgtttctggcttgcgtcctggtcctgattgtggatgccgtcctgcaactgtcccccctcctgtttatcctcgccattttgacagagtggggctccggcaataggacatacggacccgtcttcatgtgcctcggcggactgctcaccatggccgctgacaatggccctcaagaccccgataacacagacgataacggaccccatgaccctctgcctcacaatccctccgactccgccggagagtccatcgtctgctatttcatggtgtttctgcaaacccatatctttgccgaagtgctcaaggatgccattaaggatctggtcatgacaagcacagtggtcaccgctaccggactggctctgtccctgctcctgctcgccgctgtggctagctcctacgctgccgctcagagaaagctcgccgctatgggaagcctcgagatggtgcctatgggagccggaccccctagccctggcggagaccctgacggatacgatggcggaaacaatggcgctgccgctgagggagacgatggcgatgacggagacgaaggcggagacggagacgaaggcgaagagggacaggaagccgctctgacacccgtcaccgtcctgacagccgtcgtgacattctttgccatttgcctcacctggaggattgaagacccccctttcaatagcctcgacgatagctcccacgaaagcgatagcaatagcaatgagggaaggcatcacctcctggtcagcggagccggagacggaccccctctgtgtgccgctatgtccgacgaaggccctggcacaggccctggcaatggcctcggcgaaaagggagacacaagcggacccgaaggctccggcggaggcggacccgatggcgaacccgatgtgcctcccggagccattgagcaaggccctgccgatgaccctggcgaaggccctagcacaggccctctggctgccgtcgcctccagctatgccgctgcccaaaggaaactgctcacccctgtgacagtgctcaccgctgtggtcacctttttcgctcagcatgacggaaacgatggcctcccccctcccccttactcccccagagacgatagctcccagcatatctatgaggaagccggaaggggagtgctcgtgatgctggtcctgctcatcctcgcctataggagaaggtggaggagactgacagtgtgtggcggaatcatgttcctcgcctgtggcggaagcaaaacctccctgtataacctcaggagaggcacagccctcgccattccccaatgcagactgacacccctcagcagactgcctcaccatctgctcgtgtccggcgctggcgatggccctcccctctgctcccagaatctgggagcccctggcggaggccctgacaatggccctctgggagagaaaggcgatacctccggccctgagggaagcggaggctccggccctcagagaaggggaggcgataaccatggcagaggcagaccctcccccggaggcgatcccgatggctatgacggaggcaataactcccagcatccctccgcctccggctccagcggaaacacacccacaaggacaaccgatgagggaacctgggtggctggcgtcttcgtctacggaggctccaagacaagcctctacaatctgagaaggggaaccgctggcgatgacgatgaccctcacggacccgtccagctcagctattacgatgccgctctgctctggacactggtcgtgctcctgatttgctccagctgtagctcctgccctctgtccaagattctgctcgccagactgtttctgtatttcgtcctgtggctgtccagccctggcggactgggaaccctcggcgctgccctcctgacactggctgccgctctggctctgctcgcctccacctatggccctgtgtttatgtgtctgggaggcctcctgacaatggtcgccggagccgtctggctcaccgtcatgtccaacacactgctcagcggaagccctcccagaaggcctccccctggcagaaggcctttctttcaccctgtgggaagatccgaggctgactatttcgaataccatcaggagttccatcccgtcggcgaagccgattactttgagtatcaccaagagggaggccctgacggagagcctgacgtcccccctggcgctatcgaaagggatctgctctgccctctgggaggcctcggcctcctgctcctgatgatcacactgctcctgattgccctctggaatctgcatggccaaaggggacagggagacggaggcagaaggaaaaagggaggctggttcggaaagcataggggacagggaggctccaaccctaagtttgagaatatgtccgactggaccggaggcgctctgctcgtgctctactccttcgctctgatgctgattatcattatcctcatcattttcattttcagactgggagccgtcaccgtcgtgtccatgacactgctcctgctcgcctttgtgctttggctcagctcccccgggggcctcggcacactgggagtgctcgtgctcatcgtggacgctgtgctccagctcagccctctgctcggcgctgtgacagtggtcagcatgaccctcctgctcctggctccctatgaagacccctattggggaaacggagacagacactccgactatcagcctctgggaacccaagaccaaagcctctacctcggcctcctgctcctgctcctgctcgccctcctgttttggctctacattgtgatgagcgattggacaggcggagccctcctggtcctgtatagctttgagcatcaccatgacgatagcctcccccatccccaacaggctaccgatgactccagccatgagtccgactccaactccaacgaaggcagactggctatccctcagtgtaggctcacccctctgtccaggctcccctttggcatggcccctggccctggccctcagcctggccctctgagaatgattaccctcctgctcatcgctctgtggaacctccacggacgggctctgtatctgggaatcgtcctgtttatctttggctgtctgctcagggatgactccagccaacacatttacgaagaggctggcagaggctccatgaatcccgtctgccttcccgtcatcgtcgccccttacctcgacgatggcgtggacctccccccttggcttccccctatggtcgagggagccgctgccgaaggcgatgacggagacgatggcgatgagggaagccaataccctagcgctagcggaagctccggcaatacccctacccctcccaatgacgaagagagagagtccaacgaagggcctccccctggctccggctccggccctaggcatagggatggcgtcaggagaccccaaaagagaccctcctgcattggctgtaagggaaccgctgccttcgctctgtttggcgtcatcagatgctgtaggtattgctgttactattgcctcaccctcgagtccgaggaaaggcctcccacaccctatttcgctgaggtcctgaaagacgctatcaaagacctcgtgatgaccaaacccgctcccacatgcaatatcagagtgacagtgtgtagctttccccctaacgatgaggaaagggaaagcaatgaggaaccccctcccccttacgaagacccttactggggcaatggcgataggcatagcgatgccattctgatttggatgtactatcacgggcccagacacacagacgaacaccatcacgatgactccctgcctcaccctcagcaagccacaatctgtctgacatggagaatcgaagaccctccctttaactccctgctcttcgctctgctcgccgctgccggaggcgtccagggaatctatcagggacccgctgacgatcccggagagggaccctccaccggacccagaggccaaggcgatggcggaaggagaaagaaaggcggatggtttctgattctgggaaccctcaacctcaccacaatgtttctgctcatgctcctgtggaccctcgtggtcctgctcatctgtagctcctgctccgccgctatggaaagggatctggaaaggggaccccctggccctcccagaccccctctgggaccccctctgtccagctccatcggactggctatcgctgagggactgagagccctcctggctaggtcccacgtcgagagaaccacagacgaaggcacatgggtcgccggagtgtttgtgtatgccctctacctcggcattgtgctcttcattttcggatgcctcctggtcctgggactgtggatctatttcctcgagattctgtggaggctcagccaaaacctcggcgctcccggaggcggacccgataacggaccccaagaccctgacaataccgatgccgctaacgatggcggaccccctaacctcaccgaagaggtcgccaataagggaggcgataggggaccccctagcatgaccgatggcggaggcggatggtggaccctcctggtggacctcctgtggctgctcctgtttatggctatcctcatctggatgtattaccatggccctaggcataccgatgtgctcggcctctggatttactttctggaaatcctctggagactgggagccacactgtggcagctcctggctttcattctggctttctttgccctcgccctcctgctcctggctagcgctctgattgccggaggctccatcctccagacaaactttaagtccctgtccagcacagagtttttcggaatggctcccggacccggaccccaacccggacccctcagggaaagcattgtgtgttactttatggtcttcctccagacacacattggcagaggcagaggcagaggcggaggcagacccggagcccctggcggaagcggaagcggacccagacacagagacggagtgagaaggcctagctgtcccctcagcaaaatcctcctggctaggctcttcctctacgctctggctctgctcctgctcgcctccgccctcatcgctggcggagaccctcacctccccacactgctcctgggaacctccggctccggcggagacgatgacgatccccatggccctgtgcaactgtcctactatttctggctggctgccattgccgctagctgtttcacagcctccgtgtccaccgtcgtgacagccacaggcctcgccctcagcctcctgctcctgtttgccctcctggctgccgctggcggactgcaaggcatttacgtcctggtcatgctcgtgctcctgattctggcttacagaaggagaggcaaacacagaggccaaggcggaagcaatcccaaattcgaaaacattgccgaaggcctcagggctctgctcgccagaagccatgtggaaagcattctgcaaaccaatttcaaaagcctcagctccaccgagttcattcccaatctgttttgcatgctgctcctgattgtggctggcattgccgctctgctcaccctcgccgctgccctcgccctcctggctagcctcatcctcggcacactgaatctgacaaccatgttcctcctgatgagcgcttggattctgacagccggattcctcatctttctgattggctttgccctcttcggagtgattaggtgttgcagatactgttgctatctggctatcattctgctcatcattgccctctacctccagcaaaactggtggacactgctcgtggatctgctctggctcctgctcttcatgtgatctagagaattcgcc